(19) United States
(12) Patent Application Publication
(10) Pub. No.: US 2024/0117305 A1
(43) Pub. Date: Apr. 11, 2024

(54) THREE-DIMENSIONAL PRINTABLE BIOCOMPATIBLE PHOTOPOLYMERIZABLE INK WITH SPATIALLY CONTROLLED DUAL PROTEIN MOTIFS FOR CO-CULTURE FUNCTIONALITY

(71) Applicant: Rowan University, Glassboro, NJ (US)

(72) Inventors: Peter Adam Galie, Moorestown, NJ (US); Louis Salvatore Paone, Glassboro, NJ (US)

(21) Appl. No.: 18/216,337

(22) Filed: Jun. 29, 2023

Related U.S. Application Data
(60) Provisional application No. 63/413,729, filed on Oct. 6, 2022.
(60) Provisional application No. 63/420,214, filed on Oct. 28, 2022.
(60) Provisional application No. 63/521,227, filed on Jun. 15, 2023.

(51) Int. Cl. C12N 5/079; C09D 7/63; C09D 171/02

(52) U.S. Cl. CPC C12N 5/0622; C09D 7/63; C09D 171/02; C12N 2513/00; C12N 2537/10

(57) ABSTRACT

A three-dimensional biocompatible photoink useful for three-dimensional printing includes a biocompatible polymer having at least one covalently bonded photoink. The biocompatible photoink can suitably include a photoinitiator, a photo-absorber and a crosslinker and, in some embodiments, may include live tissue cells and an RGD peptide protein.

### CROSS-REFERENCE TO RELATED APPLICATIONS

This application claims the benefit of priority, under 35 U.S.C. § 119(e), of U.S. provisional patent application Nos. 63/413,729, filed Oct. 6, 2022, 63/420,214, filed Oct. 28, 2022, and 63/521,227, filed Jun. 15, 2023, the entire disclosure of each of which is hereby incorporated herein by reference.

### COLOR DRAWINGS

This patent or application file contains color drawings. Copies of this patent or patent application publication with color drawing(s) will be provided by the Office upon request and payment of the necessary fee.

### FIELD OF THE INVENTION

The present disclosure relates to a three-dimensional (“3D”) printable biocompatible polymer ink (herein “photoink”) having covalently bonded peptides which can provide spatially controlled dual protein motifs for co-culture functionality.

### DISCUSSION OF RELATED ART

Various photoinks have been used in 3D printing systems. U.S. Pat. No. 11,370,928, issued to Moussa, describes a photoink that includes 20-60% by weight of an oligomeric curable material, 10-50% by weight cyclocarbonate methacrylate monomer, and 0.1-5% by weight of a photoinitiator, based on the weight of the ink. U.S. Pat. No. 11,384,250, issued to Moussa, describes a photoink that includes up to 80% by weight of an oligomeric curable material, up to 10% by weight of a photoinitiator, and up to 10% by weight of one or more additional materials, based on the weight of the ink.

There are presently no 3D-printable photoinks that allow the functionalization of different cell binding motifs such as covalently bonded peptides. Moreover, presently available in vitro photoink systems provide no control over cellular adhesion. For example, endothelial adhesion to an extracellular matrix is an important regulator of vascular homeostasis. Yet current perfusable vascular systems rely on non-specific bonding of the endothelium to protein-based hydrogels or chemically modified biocompatible polymers. There is a need or desire in the 3D printing industry for photoinks that provide the capability to covalently attach peptide motifs that better mimic the in vivo microenvironment.

Additionally, commercially available 3D printed scaffolds are becoming more prevalent. There is a need or desire in the 3D printing industry for photoinks that provide improvements in these 3D printed scaffolds.

### SUMMARY OF THE DISCLOSURE

The present disclosure addresses the foregoing issues by providing a photoink that has the capability of being 3D printed using digital light-processing photopolymerization chemistry. The present disclosure is directed to a 3D-compatible photoink that enables the functionalization of different cell binding motifs such as covalently bonded peptides. The 3D printable photoink provides the ability to covalently attach peptide motifs that better mimic the physiological environment, and which can be used with 3D-printed scaffolds.

The use of digital light processing photopolymerization chemistry enables the photoink to print different topographies and networks that are biocompatible and functionally driven with cell binding cues. The present disclosure also provides for tunable and modular incorporation of cell-specific peptides to control fate in 3D-printed cell-seeded scaffolds.

In one embodiment, the present disclosure is directed to a 3D-printable photoink that includes a biocompatible polymer having covalently bonded protein peptides. In one embodiment, the biocompatible polymer can be polyethylene glycol norbornene, for example, 20 kDa 8-arm PEG-Norbornene. The polyethylene glycol norbornene can be present in a molar concentration of about 1 to about 10 millimoles per liter (mM), or about 1.5 to about 5 mM, or about 2.9 mM in the photoink solution.

In another embodiment, the biocompatible polymer can be hyaluronic acid norbornene. The hyaluronic acid norbornene can be present in a molar concentration of about 0.2 to about 2.0 moles per liter (M), or about 0.3 to about 1.5 M, or about 0.811 M in the photoink solution.

In one embodiment, the 3D-printable photoink can include a photoinitiator. The photoinitiator can, for example, include one or more of lithium phenyl-2,4,6-trimethylbenzoulphosphate (“LAP”), 1-hydroxy cyclohexyl phenyl ketone, 2-hydroxy-2-methyl-1-phenyl-1 propanone, and 2-hydroxy-1-[4-(2-hydroxyethoxy) phenyl]-2-methyl-1-propanone. The photoinitiator can be present in a concentration of about 10 to about 300 mM, or about 15 to about 200 mM, or about 20 mM to about 100 mM, or about 34 mM in the photoink solution.

In one embodiment, the 3D-printable photoink can include a photo-absorber. The photo-absorber can, for example, include one or more of tartrazine, curcumin, and anthocyanin. The photo-absorber can be present in a in a concentration of about 1 to about 5 mM, or about 1.5 to about 3 mM, or about 2.5 mM in the photoink solution.

In one embodiment, the 3D-printable photoink can include a crosslinker. The crosslinker can, for example, include one or more of be polyethylene glycol dithiol (“PEG-Dithiol”) and dithiothreitol. The crosslinker can be present in a concentration of about 3 to about 15 mm, or about 5 to about 10 mM, or about 8.07 mM in the photoink solution.

In one embodiment, the 3D-printable photoink can further include live tissue cells. The live tissue cells can, for example, be normal human astrocyte cells, or any stromal cells. The cells can be present in a concentration of about 100,000 to about 10 million cells per ml, or about 1 million to about 5 million cells per ml, in the photoink solution.

In one embodiment, the 3D-printable photoink can further include an arginylglycylaspartic acid (“RGD”) peptide protein. In one embodiment, the RGD protein can be any protein that includes the amino acid cysteine. In one embodiment, the RGD peptide protein can include the following amino acid sequence: [{Gly}{Cys}{Gly}{Tyr}{Gly}{Arg}{Gly}{Asp}{Ser}{Pro}{Gly}]. The RGD peptide protein can be present in a concentration of about 0.005 to about 1 mM, or about 0.05 to about 0.5 mM, or about 0.1 to about 0.3 mM, or about 0.13333 mM in the photoink solution.

The 3D printable photoink can include all, or less than all, or more than all of the foregoing ingredients in an aqueous solvent. The aqueous solvent can, for example, be water or distilled water, and can include a buffering agent for pH control of the photoink. The photoink can have a controlled pH of about 7.0 to about 7.7, or about 7.3 to about 7.5. One example of a suitable buffering agent is a phosphate-buffered saline (PBS). In one example, the aqueous solvent can have a 1× working concentration, which includes 137 mM NaCl, 2.7 mM KCl, 8 mM Na₂HPO₄, and 2 mM KH₂PO₄.

In one embodiment, the 3D printable photoink can include the following ingredients:
a biocompatible polymer having at least one covalently bonded peptide;
a photoinitiator selected from the group consisting of lithium phenyl-2,4,6-trimethylbenzoulphosphate, 1-hydroxy cyclohexyl phenyl ketone, 2-hydroxy-2-methyl-1-phenyl-1 propanone, 2-hydroxy-1-[4-(2-hydroxyethoxy) phenyl]-2-methyl-1-propanone, and combinations thereof;
a photo-absorber selected from the group consisting of tatrazine, curcumin, anthocyanin, and combinations thereof; and
a crosslinker selected from the group consisting of polyethylene glycol dithiol, dithiothreitol, and combinations thereof.

In another embodiment, the 3D printable photoink can include the following ingredients:
a biocompatible polymer having at least one covalently bonded peptide;
a photoinitiator;
a photo-absorber;
a crosslinker;
live tissue cells; and
an RGD protein sequence.

These and other embodiments will become further apparent from the following Detailed description, read in conjunction with the accompanying Figures.

### BRIEF DESCRIPTION OF THE FIGURES

An understanding of the following description will be facilitated by reference to the attached drawings, in which:

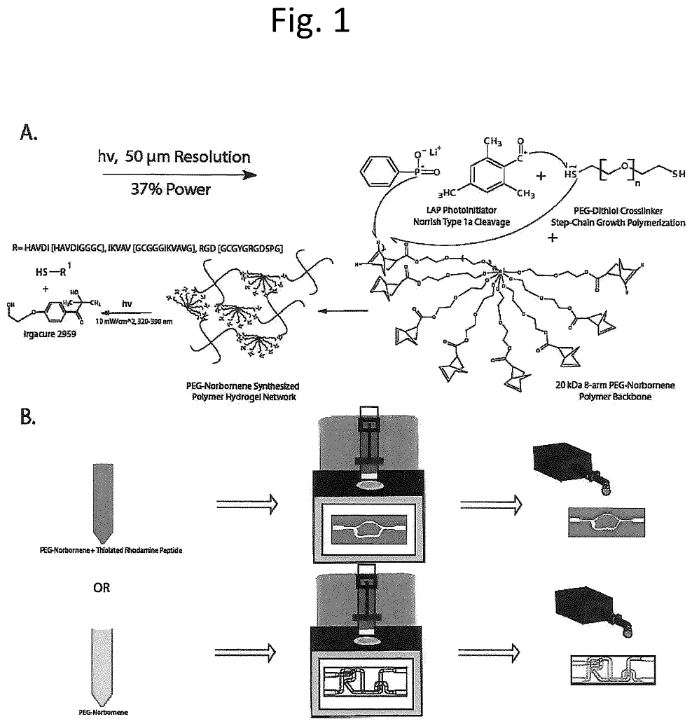

FIG. 1A is a reaction schematic of Norrish Type 1 a cleavage step growth photopolymerization of 20 kDa 8-arm PEG-norbornene using PEG-Dithiol crosslinker, followed by synthesis of photopatterning thiolated peptide motifs using Irgacure 2959.

FIG. 1B is a schematic of two distinct 3D bioprinting workflows for material functionalization of various peptide motifs, yielding partially controlled covalently bound peptides in the bulk as well as lining the inside of the channels.

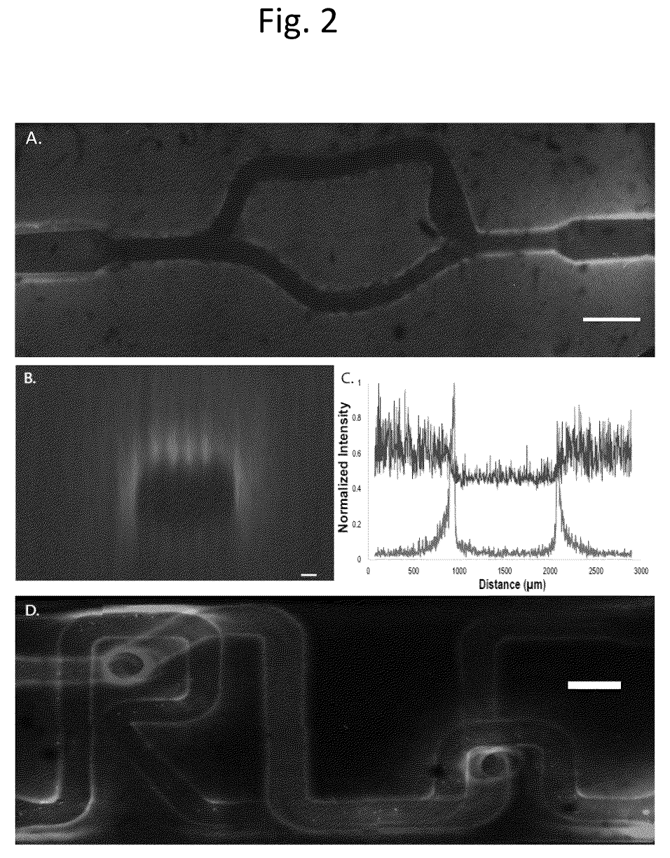

FIG. 2A is a 4× confocal stitched image of rhodamine peptide (GCDDDK-Rhod) printed inside the bulk PEG-norbornene hydrogel and green fluorescent peptide (RADA16-GFP) functionalized to the walls of the bifurcation geometry.

FIG. 2B is a cross section of the inlet displaying full coverage of RADA16-GFP covalently bound to the walls in 3D.

FIG. 2C is a fluorescent peptide intensity plot over the cross section in FIG. 2B for both green and red fluorescent peptide (GFP, RFP).

FIG. 2D is a proof-of-concept for multiple and selective peptide functionalization in complex, interpenetrating topologies.

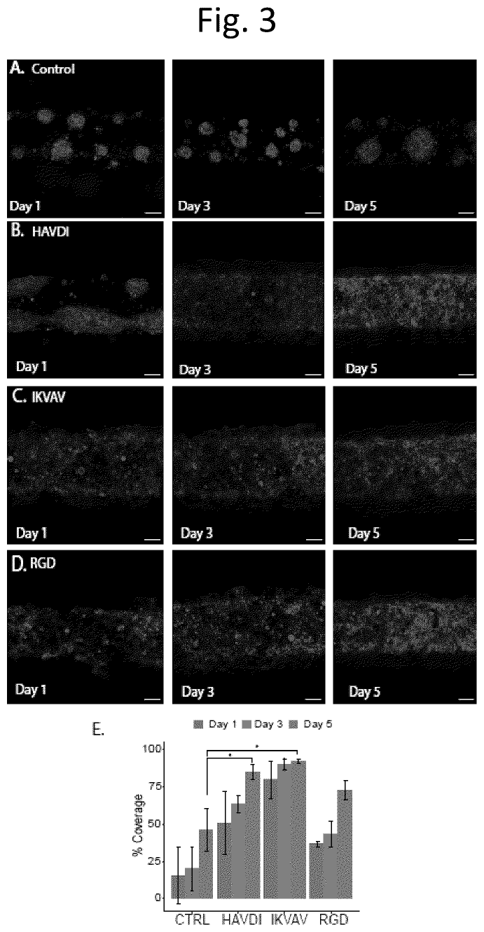

FIGS. 3A-3E show 10× time course images of P23 transfected LifeAct HCMEC/D3 seeded cells at 50M/mL captured on days 1, 3, and 5 in straight 650 μm channels with covalently bound peptide motifs. FIG. 3A: Control, no peptide functionalized to the wall of the channel. FIG. 3B: HAVDI, derived from N-Cadherin. FIG. 3C: IKVAV, derived from laminin. FIG. 3D: RGD, derived from multiple ECM proteins, adhered to the walls of the vessel. FIG. 3E: Percent cell coverage quantification within the vessel across 5 days for each peptide condition (*=p<0.05). Scale Bars=100 μm.

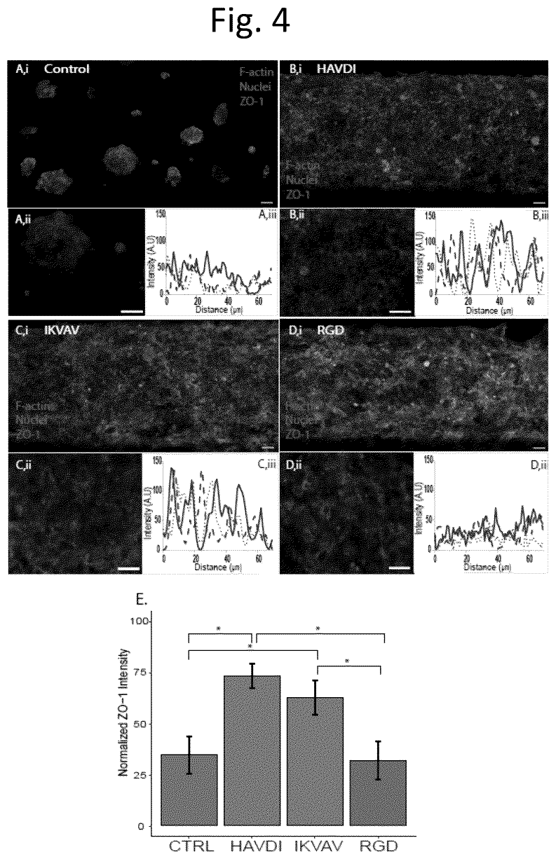

FIGS. 4A-4D show Day 5 static cultured vessels stained for ZO-1 within each peptide condition. (i) Composite image of transfected F-actin, cell nuclei (Hoesch), and ZO-1 (2-antibody immunocytochemistry). (ii) Isolated ZO-1 stain with cross section intensity profile plots across cell-cell junctions (iii).

FIG. 4E shows a normalized root mean square ZO-1 intensity quantification (*=p<0.05). Scale Bars=50 μm.

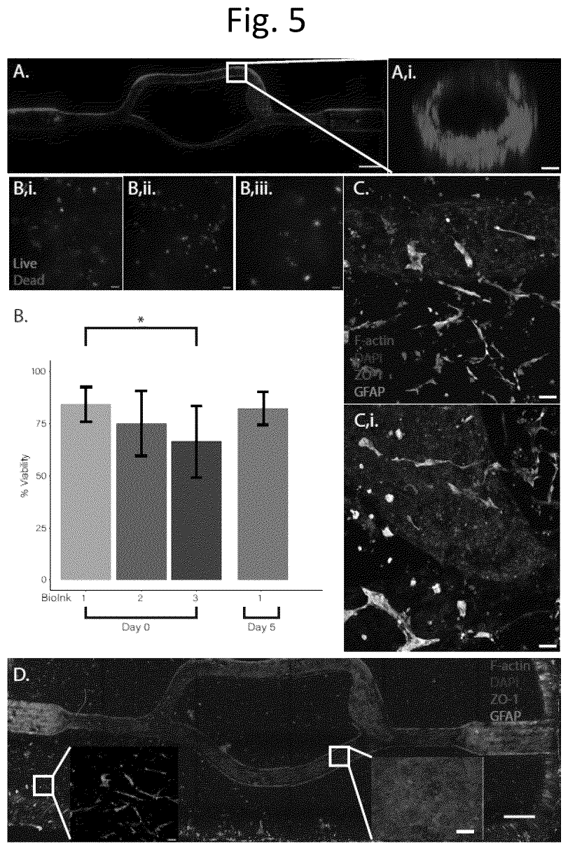

FIG. 5A shows a stitched bifurcation vessel with P23 LifeAct HCMEC/D3 cells lining the walls of the channel with RGD peptide (Scale Bar=1 mm) and (A,i.) cross-section of endothelium in the branch (Scale Bar=50 μm).

FIG. 5B shows a Live/Dead assay of printed astrocytes with different formulations (Photoink formulations 4, 5 and 6 below) and viability after 5 days in culture,

FIG. 5C includes composite images showing co-culture of endothelial cells and astrocytes in different locations of the 3D-printed vessel model (Scale Bars=100 μm).

FIG. 5D shows Stitched z-stack scan (Scale Bar=1 mm) of entire bifurcation showing astrocytes in the bulk and endothelium coverage with ZO-1 localization (Scale Bars=100 μm). Insets show higher magnification of astrocytes (left) and endothelial cells (right).

### DETAILED DESCRIPTION

The present disclosure is directed to a new and improved three-dimensional biocompatible photoink, as descried above and in the accompanying claims. The biocompatible photoink included a biocompatible polymer having at least one covalently bonded peptide. Suitable covalently bonded peptides include, but are not limited to, polyethylene glycol norbornene (herein “PEG-norbornene”) and hyaluronic acid norbornene. On example of a polyethylene glycol norbornene is 20 kDa 8-arm PEG-norbornene.

The biocompatible polymer and other ingredients described below can be present in an aqueous solution, which can be a pH-controlled aqueous solution. The aqueous biocompatible photoink solution can have a controlled pH of about 7.0 to about 7.7, or about 7.3 to about 7.5. One example of a suitable buffering agent is a phosphate-buffered saline (PBS). Other suitable buffering agents can also be used for the aqueous solution.

When PEG-norbornene is used as the biocompatible polymer, it can be present in a concentration of about 1 to about 10 millimoles per liter (mM), with all concentrations described herein based on one liter of photoink solution. Suitably, the PEG-norbornene can be present in a concentration of about 1.5 to about 5 mM, or about 2 to about 4 mM, or about 2.9 mM in the photoink solution. When hyaluronic acid is used as the biocompatible polymer, it can be present in a concentration of about 0.2 to about 2.0 M (moles per liter), or about 0.3 to about 1.5 M, or about 0.5 to about 1.0 M, or about 0.811 M.

In addition to the biocompatible polymer, the three-dimensional biocompatible photoink can also include one, more than one, or all of the additional ingredients described above, and can include them in any suitable or workable combinations and amounts. For example, the three-dimensional biocompatible photoink can include any one, or more than one, or all of a photoinitiator, a photo-absorber, a crosslinker, live tissue cells, and an RGD protein sequence. Specific examples of three-dimensional biocompatible photoink compositions are described below. These and other biocompatible photoink compositions having the ingredients described herein are deemed to be within the scope of the present disclosure.

In one embodiment, a three-dimensional biocompatible photoink can be provided that does not include live cells or an RDG peptide protein. Three exemplary photoink compositions using PEG-norbornene as the biocompatible polymer are provided in Table 1 below. All three compositions were provided in an aqueous solution buffered to a pH of 7.4 using 1×PBS.

TABLE 1
Exemplary Photoink Compositions, No Live Cells

| | PEG- | | PEG- | |
|---|---|---|---|---|
| Photoink | Norbornene, | LAP, | Dithiol, | Tartrazine, |
| Formulation | mM | mM | mM | mM |
| 1 | 2.941 | 34 | 8.07 | 2 |
| 2 | 2.941 | 25.5 | 8.07 | 2 |
| 3 | 2.941 | 17 | 8.07 | 2.5 |

In another embodiment, a three-dimensional biocompatible photoink can be provided including both live stromal cells and an RGD peptide protein. Advantages of this embodiment are that it provides co-culture modality to the photoink and a better mimic of live tissue. Three exemplary photoink compositions using PEG-norbornene as the biocompatible polymer are provided in Table 2 below. The RGD peptide protein included an amino acid cysteine having the following amino acid code sequence:
[{Gly}{Cys}{Gly}{Tyr}{Gly}{Arg}{Gly}{Asp}{Ser}{Pro}{Gly}].

TABLE 2
Exemplary Photoink Compositions with Live Cells

| | | | | | RGD | |
|---|---|---|---|---|---|---|
| | | | | | Protein, | |
| | | | | | Sequence | Stromal |
| Photo | PEG- | | PEG- | | [GCGYGRG | Cell |
| ink | Norbornene, | LAP, | Dithiol, | Tartrazine, | DSPG], | Concentration |
| Formulation | mM | mM | mM | mM | mM | (Million/ml) |
| 4 | 2.941 | 34 | 8.07 | 2 | 0.13333 | 1 |
| 5 | 2.941 | 25.5 | 8.07 | 2 | 0.13333 | 1 |
| 6 | 2.941 | 17 | 8.07 | 2 | 0.13333 | 1 |

Exemplary printing conditions for the foregoing ink formulations and those that follow are provided below. In another embodiment, a three-dimensional biocompatible photoink can be provided that either does or does not include live cells and an RGD peptide protein and uses hyaluronic acid norbornene instead of PEG-norbornene as the biocompatible polymer. One exemplary photoink compositions that utilizes hyaluronic acid norbornene are shown in Table 3. As described above and represented below, the optimal concentrations and ranges of hyaluronic acid norbornene are higher that for PEG-norbornene. The remaining ingredients can be provided using similar concentrations.

TABLE 3
Photoink Composition With HLA-norbornene

| | Hyaluronic | | | |
|---|---|---|---|---|
| | Acid- | | PEG- | |
| Photoink | Norbornene, | LAP, | Dithiol, | Tartrazine, |
| Formulation | mM | mM | mM | mM |
| 7 | 811 | 34 | 8.07 | 2 |

As explained above, various photoinitiators can be included in the three-dimensional biocompatible photoink. Table 4 provides a detailed description of three exemplary photoinitiators that have been found suitable for use with the inventive photoink composition.

TABLE 4
Compatible Photoinitiators for the Photoink

| | | | | | UV/VIS |
|---|---|---|---|---|---|
| Name | Chemical Identity | Mechanism | Chemical Class | Appearance | Peaks, nm |
| IRGACURE | 1-Hydroxy-cyclohexyl- | Norrish | α-Hydroxyketone | white to off-white | 246, 280, |
| 184 | phenyl-ketone | Type 1-α | | crystalline powder | 333 |
| | | Cleavage | | | |
| DAROCUR | 2-Hydroxy-2-methyl- | Norrish | α-Hydroxyketone | clear, light yellow | 245, 280, |
| 1173 | 1-phenyl-1-propanone | Type 1-α | | liquid | 331 |
| | | Cleavage | | | |
| INGACURE | 2-Hydroxy-1-[4-(2- | Norrish | α-Hydroxyketone | off-white powder | 276 |
| 2959 | hydroxyethoxy)phenyl]- | Type 1-α | | | |
| | 2-methyl-1-propanone | Cleavage | | | |

Table 5 shows an exemplary photoink formulation 8 in which dithiothreitol was used for the crosslinker instead of PEG-dithiol. The concentration of PEG-norbornene was also lower that in formulations 1-3 above, but still within the range of the inventive formulation.

TABLE 5
Photoink Formulation Using DTT Crosslinker

| | PEG- | | Dithiothreitol | |
|---|---|---|---|---|
| Photoink | Norbornene, | LAP, | (DTT), | Tartrazine, |
| Formulation | mM | mM | mM | mM |
| 8 | 1 | 8 | 8 | 1.5 |

Table 6 shows an exemplary photoink formulation 9 in which dithiothreitol was used for the crosslinker instead of PEG-dithiol and curcumin was used instead of tartrazine for the photo-absorber.

TABLE 6
Photoink Containing DTT and Curcumin

| | PEG- | | | |
|---|---|---|---|---|
| | Norbornene, | | Dithiothreitol | |
| Photoink | mM | LAP, | (DTT), | Curcumin, |
| Formulation | (Molarity) | mM | mM | mM |
| 9 | 1 | 8 | 6 | 2.5 |

Mixing of the photoink formulations can be accomplished using the following procedure. First, the biocompatible polymer having at least one covalently bonded peptide can be added to water or a pH-buffered aqueous solution and the mixture can be sonicated to solubilize the biocompatible polymer. The sonification can be performed using any suitable sonicator, for example a Branson 1800 Sonicator available from Branson Ultrasonics Corporation in Buffalo Grove, Illinois. The sonicator can operate at a frequency of about 40 KHZ, or any suitable frequency, for a solution temperature of about 22° C. Once the biocompatible polymer has been solubilized in the aqueous solution, the remaining ingredients, namely the photoinitiator, photo-absorber, crosslinker and, if used, live tissue cells and RGD protein sequence can be added to the solution and mixed.

The three-dimensional biocompatible photoink provides the capability to precisely tune cell-matrix interactions within 3D-printed scaffolds. There are several foreseeable uses of this technology including the fabrication of in vitro model systems to study drug delivery and efficacy as well as transplantable scaffolds for regenerative medicine. The scaffolds exhibit modular and tunable cell adhesion peptide motifs, making this approach broadly applicable to multiple organ systems and tissues. The 3D-printed in vitro systems have commercial applicability to both pharmaceutical companies and academic research laboratories. Additionally, 3D-printed scaffolds for regenerative medicine can improve current strategies to create organ-level transplants, which would hold tremendous market value.

According to the present disclosure, endothelial adhesion to the extracellular matrix is an important regulator of vascular homeostasis. By contrast, conventional perfusable vascular systems rely on non-specific binding of the endothelium to natural protein-based hydrogels or chemically modified biocompatible polymers. In one embodiment, the present disclosure is directed to a strategy to functionalize multiple peptide motifs within a single, DLP-printed network topology. This approach allows selective clicking of multiple peptides within the bulk of hydrogels featuring interpenetrating channels and into the lining of these channels. Peptides derived from proteins mediating cell-extracellular matrix (“ECM”), for example, RGD and Ile-Lys-Val-Ala-Val (“IKVAV”) and cell-cell, for example, Histidine-Analin-Valine-Aspartate-Isoleucine (“HAVDI”) adhesions can be used to mediate endothelial cell attachment, spreading, and coverage.

Endothelial spreading and coverage assays indicate that HAVDI and IKVAV significantly improve endothelialization compared to other motifs. Moreover, HAVDI and IKVAV-lined channels increase zonula occulends-1 (“ZO-1”) localization to cell-cell junctions of cerebral endothelial cells, indicative of tight junction formation. An RGD-lined network topology designed to induce flow separation reveals no significant difference in ZO-1 localization in response to fully developed and disturbed flow, suggesting that tuning cell attachment modifies the endothelial response to fluid shear stress. In addition to creating a new platform to interrogate endothelial mechanobiology, this broadly applicable approach enables a pathway to fabricate vascularized scaffolds informed by native cell-matrix interactions.

In accordance with the present disclosure, the composition and structure of the extracellular matrix influences endothelial cell function and flow mechanosensing by dictating integrin binding and subsequent signaling transduction. The endothelium binds to a laminin-rich basement membrane that exhibits a host of peptide motifs known to interact with integrins on the basal surface of the cell. Previous studies have demonstrated that laminin-associated motifs including RGD and IKVAV can be used to create engineered microenvironments that mimic native cell-matrix interaction and support endothelial growth and tubulogenesis. RGD in particular has been used to facilitate endothelial attachment in both two and three-dimensional systems. The RGD motif binds to a wide array of integrin isoforms, perhaps the most well-characterized being αVβ3, that are known to mediate the endothelial response to shear stress. Studies suggest that tuning cell-matrix interaction alters endothelial mechanotransduction of fluid flow, though there has yet to be a strategy to interrogate this directly.

In one embodiment, fabricating engineered microenvironments to mimic cell-ECM interactions involves radically driven thiol-ene based reactions. Covalently driven binding of extracellular matrix-associated peptides can be achieved by utilizing specific biomaterials with a high degree of functionality. Briefly, near UV photochemical Norrish type I-α cleavage drives selective thiol-ene peptide functionalization to the polymer backbone. These include both synthetic, PEG-based formulations as well as naturally derived biomaterials like hyaluronan. Different reaction schemes for selective peptide modification include Huisgen cycloaddition via PEG tetra-azide in addition to thiol-ene mediated polymerization via PEG norbornene. Selective and spatial control of peptide binding using this approach has been demonstrated by many groups in both two and three-dimensional topologies. These techniques use click chemistry to selectively bind peptides to a desired region within the hydrogel framework.

The development of digital light processing (DLP) provides the capability to fabricate complex topologies within photopolymerizable thiol-ene based chemistries but has been limited to self-assembling monomers that do not mediate covalent biochemical cues, since these polymers form due to the backbone binding to itself. Gelatin methacrylate (GeIMA) is frequently incorporated within these polymers to facilitate endothelial cell attachment. Yet, GeIMA relies on non-specific integrin binding and furthermore, is derived from a xenographic source. PEG-norbornene is an excellent candidate for DLP for its versatility with incorporating multiple domains. Step-growth chain polymerization, which typically uses a crosslinker to bind two monomer units together, frees up additional thiol-ene binding domains. Moreover, higher n-domains provides faster reaction kinetics and ultimately low exposure times for live cell printing. One embodiment of the present disclosure includes a process to print interpenetrating networks in PEG-Nor hydrogels using DLP, then to spatially pattern peptide motifs in a secondary cross-linking process. This approach provides a platform to interrogate the effect of cell-matrix interactions on endothelial spreading and lumen formation within complex vascular topologies. Moreover, shear stress is applied to the vessel lumen to test the hypothesis that the peptides lining the channels affect fluid flow mechanotransduction.

### Examples

Examples of the disclosure were prepared according to the following methods.

3D Bioprinting

3D printed hydrogel networks were fabricated using a Lumen X+ digital light processing printer (CellInk). Briefly, computer-aided design (CAD) files devised in Solidworks were uploaded onto the Lumen X+ bioprinter in STL format. Lyophilized 20 kDa, 8-arm poly-ethylene glycol norbornene polymer (JenKem Technologies) was reconstituted in phosphate buffered saline (PBS). After solubilization, lithium phenyl-2,4,6-trimethylbenzoylphosphinate (LAP, Allevi) was added to poly-ethylene glycol dithiol crosslinker (PEG-Dithiol, JenKem Technologies), and a photoabsorber, tartrazine (Millipore Sigma). The photoink was added to a PDMS-lined P100 dish and printed with 50-μm step resolution in the z-axis. Synthesized gels were washed in PBS 2× for 30 minutes to remove unpolymerized polymer and excess reagent.

For the photoink formulations 1 to 7 shown in Tables 1, 2 and 3, digital light processing (DLP) was accomplished using laser power of 16.66 mW/cm². The 405 nm exposure times were 2.25 sec. for formulations 1 and 4, 2.75 sec. for formulations 2 and 5, 3.0 sec. for formulations 3 and 6, and 1.25 sec. for formulation 7. The burn-in multipliers, representing the time-multiplier for the first layer of gel, were 3× for formulations 1 to 6 and 5× for formulation 7.

For the photoink formulations 8 and 9 shown in Tables 5 and 6, DLP was accomplished using laser power of 40.13 mW/cm². The 405 nM exposure times were 24 sec. for formulation 8 and 18 sec, for formulation 9. The burn-in multipliers were 3× for both formulations.

Functionalizing Cell Binding Motifs

Radicalized driven thiol-ene reaction schemes were utilized for functionalizing biomaterials. Several thiolated peptide cell binding motifs were functionalized to the walls of the printed geometry. Fluorescent labeled peptides were first synthesized via peptide synthesizer. Rhodamine labeled peptide (GCDDDK-Rhod) and green fluorescent protein labeled peptide (RADA16-GFP) were both synthesized to demonstrate selective and controlled protein functionalization within different complex geometries. Lyophilized RFP and GFP were both brought to room temperature and diluted to 10 mg/mL in 1×PBS. The bifurcation geometry print included RFP peptide in the bulk solution at 1:1000, while 300 μL of GFP was flowed into the channel at 10 μg/mL and hit with mW/cm² for 180 seconds. Process was repeated in the same manner for channel functionalization of the ‘RU’ geometry. Similarly, HAVDI(HAVDIGGGC), derived from N-Cadherein and RGD(GCGYGRGDSPG), derived from multiple ECM proteins, were synthesized and purchased from Genscript. IKVAV(GCGGGIKVAVG), a laminin derived motif, was fabricated using a peptide synthesizer. 5 mg/mL Irgacure 12959 was diluted to 0.5 mg/mL in PBS and thiolated peptide was added to a final concentration of 5 mM. 50 μL of peptide solution was injected into the channel and 10 mW/cm² was radiated onto surface of gel for 60 seconds. This process was repeated after inverting the gel to assure full coverage of the peptide onto the surface of the channel. Gels were then washed out 2× for 30 minutes in PBS to remove any unbound peptide.

Endothelial Cell Seeding

P23 hCMEC/D3 endothelial cells (with and without transfection with RFP-LifeAct) were cultured until 90% confluent, trypsinized, and suspended at a concentration of 50M/mL for hydrogel cell seeding. Hydrogels were seeded with 22 μL of 12.5 M/mL and placed face up at the bottom of six well plate with 1 mL of EGM-2 incubated at 37° C./5% CO₂. The cell seeding technique included 90° rotations every 30 minutes with additional seeding every hour for 4 hours. Following initial seeding, 8.8 μL of 12.5 M/mL HCMEC/D3 were injected into the bifurcation geometry for vessel coating. After 4 hours, hydrogels were kept in static culture for 4-5 days in EGM-2.

Vessel Perfusion

Vessels cultured in static conditions for four days were hooked up to pressure driven flow using an Arduino-controlled peristaltic pump. Gels were ported inside a perfusion chamber with 20-gauge PTFE catheter tips connected to luer locks with tygon tubing (McMaster Carr). Vessels were connected in parallel through a dampener with EGM-2 for 24 hours at a flow rate of ˜5 mL/min or 44.4 dyne/cm² shear stress in the branches and 1.68 dyne/cm² in the region adjacent to the bifurcation.

Immunocytochemistry

Following static and/or flow culture within the vessels, hydrogels were washed 3× in PBS and placed in 4% paraformaldehyde for 20 minutes. Gels were washed in PBS and a primary monoclonal antibody for zonula-occluden-1 (ZO-1) (Cell Signaling Technologies) was added at 1:250 in PBS and incubated at 4° C. for 48 hours. Gels were then washed for 10 minutes 3× in PBS on the rocker. Far red-labeled ZO-1 secondary antibody (Cell Signaling Technologies) was diluted 1:250 in PBS along with Hoesch at 2 ug/ml. Solution was added to the gels and incubated for 1 hour at 37° C. Gels were then washed and imaged on a Nikon Eclipse Ti Confocal microscope.

Quantification of Cell Coverage and Barrier Development

In order to quantify cell spreading, a z-projection was used with an arbitrary ROI to quantify the percentage of the area covered by cells. Three different ROI's were analyzed to provide an average and standard deviation. ZO-1 intensity was quantified in both static and perfused culture at different locations within the branched network. The image intensity along approximately 70-μm lines across the image plane were plotted to quantify ZO-1 localization to cell-cell junctions. Root mean squares of this data were calculated to compare ZO-1 localization between experimental groups. ImageJ was used for all image processing and analysis.

Incorporation of Live Cell Coverage and Barrier Development

Three different photoinks were used to test the viability of live cell printing normal human astrocytes (NHA). Briefly, LAP and exposure time were titrated linearly to determine how near UV exposure effected viability.

| Photoink | LAP, | 405 nm | | Tartrazine, |
|---|---|---|---|---|
| Formulation | mM | Exposure | | mM |
| 4 | 34 | 2.25 | s | 2 |
| 5 | 25.5 | 2.75 | s | 2 |
| 6 | 17 | 3 | s | 2.5 |

PEG-Dithiol and laser power were held constant.

P4 NHA's were cultured in astrocyte growth medium (Lonza) until 90% confluent in a P100 tissue culture plate, trypsinized, and reconstituted to 1M/mL in culture medium. Cells were centrifuged with media aspirated out and replaced with a custom photoink. The live cell ink was added to the preheated printing vat set to 37° C. The gel was then washed in medium on a rocker at 37° C. for 2 hours, replacing medium every 30 minutes. A live/dead assay was performed on day 0 and day 5 of NHA culture inside of the 3D printed bifurcation model. 2 μm of calcein AM and 4 μm of ethidium homodimer was added to 10 mL of PBS and the gels were incubated at 37° C./5% CO₂ for 1 hour prior to imaging.

Confocal Microscopy

Imaging was performed on a Nikon Eclipse Ti Confocal Microscope to assess cell spreading, morphology, and proliferation in 3D overtime within these microvessels. Images were taken on days 1, 3, and 5 following cell seeding.

Statistical Analysis

A two-factor ANOVA was calculated to test for significance in the two variables of time and peptide coating in the cell spreading assay. In order to test for significance in the ZO-1 localization experiments, a one-factor ANOVA was calculated for each peptide condition tested.

Results

FIG. 1 illustrates the working principle of DLP-printed PEG-Nor hydrogels. Stepwise chain-growth polymerization of poly-ethylene glycol-norbornene was used to print hydrogels with a DLP-based LumenX+ bioprinter (FIG. 1A). Subsequent photo-patterning of R-peptides using an Irgacure 2959 photoinitiator under 320-390 nm UV yielded topological integrin binding cues. Two different bioprinting workflows were demonstrated as a proof-of-concept for functionalizing multiple peptide motifs within a single topology. First, thiolated rhodamine-peptide was functionalized to the bulk hydrogel network during the print, followed by perfusing a GFP-labeled peptide into a bifurcation network topology and clicking the peptide into the walls of the vessel (FIG. 1B). This protocol was the basis of the 3D-printed blood-brain barrier model, since it demonstrated the ability to click distinct bioactive domains into the bulk of the hydrogel and the channel walls. Therefore, a peptide motif intended to facilitate astrocyte attachment could be different from a motif in the lining of the channel designed for endothelial attachment. The second workflow involved printing two separate, interpenetrating topologies and perfusing them with two peptides to demonstrate a different approach to patterning multiple peptide motifs within a single hydrogel.

Using the first workflow described in FIG. 1B, and the photoink formulation no. 1 described above, GFP-labeled peptides were flowed into and clicked into the lining of a branched topology within the hydrogel, which was printed within rhodamine-labeled peptides functionalized into the bulk (FIG. 2A). A cross section of the channel shows an evenly distributed, uniform coating of GFP-labeled peptide with an equal distribution of rhodamine-labeled peptide in the bulk (FIG. 2B). A profile plot along the x-axis of the gel overlays red and green peptide intensities across the plotted area (FIG. 2C). The result of the second post-processing workflow shown in FIG. 1B, lining two distinct, interpenetrating channels within the same bulk matrix with two different peptides, is shown in FIG. 2D. The CRU′ geometry, for “Rowan University,” includes multiple planes in the Z-axis demonstrating the ability to apply this technique within complex 3D vascular models.

FIG. 3 demonstrates the effect of peptide motif in endothelialization in DLP-printed channel using photoink formulation no. 1 described above. To study endothelial attachment and spreading in channels lined with different peptides, cylindrical voids (650 μm diameter) were generated within the PEG-Nor hydrogels. Cell coverage in channels lined with three different peptides (IKVAV, HAVDI, and RGD) in addition to a negative control (no peptide clicked to the surface) were measured over time. Cell coverage did not increase in the control condition, although spherical aggregates formed within the peptide-free cylindrical channels (FIG. 3A). IKVAV, a binding motif of the basement membrane protein laminin, exhibited no noticeable difference in vessel coverage throughout the 5-day static culture. Rather, the cells adhered immediately to the peptide and began to spread (FIG. 3B). Cells in the channel lined with HAVDI, the active sequence of the cell-cell junction protein N-cadherin, initially appeared to favor cell-cell junctions and binding to each other. However, over the course of the experiment cells began to spread and cover the walls (FIG. 3C). Finally, cells in channels lined with RGD, a common motif in multiple ECM proteins, exhibited immediate spreading on PEG-norbornene and progressed linearly overtime (FIG. 3D). Interestingly, in each of the three conditions, endothelial cells began to develop monolayer coverage even in the absence of shear stress that has previously been shown to encourage endothelialization in collagen-based hydrogels. Quantification of vessel coverage indicated that both the HAVDI and IKVAV-lined channels exhibited significantly higher coverage than the control condition in static culture at day 5 (FIG. 3E).

FIG. 4 provides an assessment of tight junction integrity within DLP-printed vessels using photoink formulation no. 1 described above. A crucial indicator of a robust and resilient barrier formation is localization of tight junction-associated proteins to the cell-cell junctions of the endothelium. Zonula occluden-1 (ZO-1), a tight junction scaffolding protein that is indicative of barrier formation in hCMEC/D3 cells, was imaged in cells cultured in the peptide-lined channels in static conditions for five days. In order to quantify the images, intensity plots of the ZO-1 staining were measured and the root mean square of the distributions were calculated. Localization at the cell-cell junctions compared to diffuse intracellular staining was indicated by increased “roughness” of the distribution. FIG. 4A indicates that the ZO-1 staining was mostly diffuse in the spheroid-like structures in the control channels. Cells in the IKVAV channels exhibited more ZO-1 localization to the cell-cell junctions (FIG. 4B). ZO-1 junctional staining was the clearest in the HAVDI-lined channels (FIG. 4C). Finally, although RGD increased cell spreading, the ZO-1 was the most diffuse in this condition (FIG. 4D). Quantification of the root mean square values indicated that the HAVDI and IKVAV conditions had significantly higher barrier formation than the RGD and control-lined channels (FIG. 4E).

The following experiments demonstrate implementing endothelial-astrocyte interaction in a 3D-printed, branched topology. The primary benefit of DLP over other methods to create channels within hydrogels is the ability to print branched, physiologically relevant topologies that mimic the complex fluid flow regimes found in vivo. Therefore, the topology showed in FIG. 2A was chosen to verify the ability to 3D-print an endothelial-astrocyte co-culture model.

Referring to FIG. 5, photoink formulations nos. 4-6 containing live cells and RGD peptide protein were tested against each other. Photoink formulation no. 4 proved best and was used to generate images C, C, I, and D in FIG. 5.

First, full coverage of the topology with only endothelial cells was demonstrated in FIG. 5A, prior to seeding the hydrogel with astrocytes. The RGD peptide motif was used to demonstrate that full channel coverage was possible even with a peptide motif that did not result in the highest ZO-1 localization to cell-cell junctions. To assure that the printing process could incorporate live cells without reducing viability, a Live/Dead assay was performed with astrocytes in the PEG-Nor formulation containing RGD peptide.

FIG. 5B shows representative epifluorescent images of the live/dead assay for each of the three photoink formulations nos. 4-6 that were tested and subsequent viability of 83% for photoink 4. Photoink 4 formulation showed the highest viability due to lower near UV exposure times despite being in the presence of a higher concentration of cytotoxic LAP. Finally, to demonstrate successful printing of endothelial-astrocyte co-culture, photoink 4 was lined with the peptide that induced the highest ZO-1 localization to the cell-cell junctions, HAVDI, and lined with endothelial cells. The channels were cultured for five days to allow full coverage of the channel. The viability of the assays at this time point was validated with Live/Dead and plotted in FIG. 5B. The channels were probed with anti-ZO-1 and anti-GFAP to show astrocytes in the bulk with endothelial cells lining the branched geometry (FIG. 5C). Finally, a stitched confocal scan was completed demonstrating the spatial control of spread astrocytes in the bulk with barrier formation of an endothelium in the lumen (FIG. 5D).

These results establish the efficacy and broad applicability of combining digital light processing (DLP) with thiolene based photoinks to tune cell-ECM and cell-cell interactions within perfusable network topologies. The workflows described here demonstrate the ability to covalently bind one peptide in the bulk of the hydrogel and a different peptide to the lining of the channel wall, as well as to click different peptides into the lining of separate, interpenetrating networks. The former is used to create a model of the blood-brain barrier with increased functionality compared to previous approaches: both in terms of its branched, network-like topology and also its flexibility in tuning bioactive domains used to facilitate both endothelial and astrocyte attachment within the model. The relatively fast reaction kinetics of the PEG-Norbornene ameliorates the cell death associated with live cell printing, as evidenced by the viability of the astrocytes after the printing, and the three-day incubation of the co-culture prior to fixation. Overall, this approach provides the tools to create a new generation of 3D blood-brain barrier models to interrogate the effects of mechanical and biochemical stimuli on the integrity of the barrier.

As demonstrated in the results, the endothelial cells are significantly affected by the bioactive domains clicked into the lining of the channels. The IKVAV and HAVDI motifs provide the most favorable microenvironment for both vessel coverage and development of barrier function in hCMEC/D3 endothelial cells. The efficacy of IKVAV could be due to the prevalence of laminin in the basement membrane, which therefore provided a more native cell-ECM interaction for the endothelial cells. Several studies have demonstrated the benefit of recreating elements of the basement membrane to increase endothelial attachment in vitro. In contrast, HAVDI is a sequence from the extracellular domain of N-cadherin, which mediates cell-cell junctions. Therefore, clicking HAVDI into the channel wall simulated cell-cell adhesion on the basal side of the endothelial cells. Unlike the IKVAV condition, the cells in the HAVDI-lined channels took several days to spread and cover the vessel wall. The molecular mechanisms underlying this dynamic response as well as the development of endothelial barrier function in peptide-lined vessels remains unresolved. Previous studies have found that N-cadherin junctions increase RhoA activation and decrease Rac1 activation, which counters a recent finding that shear stress stabilizes tight junctions by decreasing RhoA activation.

The primary advantage of printing the PEG-Nor hydrogels with DLP is its ability to fabricate perfusable, multivascular topologies that mimic in vivo vasculature. In contrast to two photon-based approaches to spatially pattern photocrosslinkable ligands within three-dimensional hydrogels, DLP produces complex vascular topologies that support physiological flow rates. However, one of the current limitations of this technology is its limited resolution: the LumenX+ printer used in these studies has a minimum printing resolution of 50-μm, which substantially limits the ability to print small caliber vessel and capillary-scale vasculature. This feature is especially important for blood-brain barrier models since the neurovascular unit is most relevant at the capillary scale. Nonetheless, the resolution used in this study is limited by the printer, not by the photoink and workflow used to generate the 3D-printed blood-brain barrier model. As printing technology improves its resolution, the same approach described here can be used to facilitate incorporation of bioactive domains to control endothelial cell and astrocyte attachment within 3D-printed topologies.

Despite the limitation in its resolution, the model described here can be used as a new platform to study the mechanobiology of the blood-brain barrier. Heterogeneous distributions of shear stress, even disturbed flow, can be applied within single network topologies to improve our understanding of the effects of complex fluid flow on the integrity of the barrier. Combining DLP with computational fluid dynamics provides a platform to correlate shear stress magnitude and gradients with changes in endothelial transcriptomics and proteomics. Moreover, the effect of peptide binding on the response of the endothelium to shear stress can also be interrogated in this platform. In addition to incorporating new peptide motifs not used in this study, equimolar ratios of multiple peptides can be covalently bound to the wall to better mimic the heterogeneous peptide sequences available to cells in the native basement membrane. This approach can be used to determine how changes in the extracellular matrix affects shear stress mechanotransduction. Finally, the advance described here is not limited to modeling of the blood-brain barrier, since there are multiple biological systems that feature prominent interaction between vascular and stromal cell types that can be modeled using this same workflow.

The embodiments described herein are exemplary, and various modifications and improvements can be made without departing from the scope of the disclosure. The scope of the disclosure is indicated in the appended claims, and all modifications that fall within the meaning and range of equivalents are embraced therein.

What is claimed is:

1. A three-dimensional biocompatible photoink, comprising a biocompatible polymer having at least one covalently bonded peptide.

2. The three-dimensional biocompatible photoink of claim 1, wherein the biocompatible polymer comprises polyethylene glycol norbornene.

3. The three-dimensional biocompatible photoink of claim 1, wherein the polyethylene glycol norbornene is present in a concentration of about 1 to about 10 mM.

4. The three-dimensional biocompatible photoink of claim 1, wherein the biocompatible polymer comprises hyaluronic acid norbornene.

5. The three-dimensional biocompatible photoink of claim 4, wherein the hyaluronic acid norbornene is present in a concentration of about 0.2 to about 2.0 M.

6. The three-dimensional biocompatible photoink of claim 1, further comprising a photoinitiator.

7. The three-dimensional biocompatible photoink of claim 6, wherein the photoinitiator is selected from the group consisting of lithium phenyl-2,4,6-trimethylbenzoulphosphate, 1-hydroxy cyclohexyl phenyl ketone, 2-hydroxy-2-methyl-1-phenyl-1 propanone, 2-hydroxy-1-[4-(2-hydroxyethoxy) phenyl]-2-methyl-1-propanone, and combinations thereof.

8. The three-dimensional biocompatible photoink of claim 6, wherein the photoinitiator comprises lithium phenyl-2,4,6-trimethylbenzoulphosphate.

9. The three-dimensional biocompatible photoink of claim 6, wherein the photoinitiator is present in a concentration of about 10 to about 300 mM.

10. The three-dimensional biocompatible photoink of claim 1, further comprising a photo-absorber.

11. The three-dimensional biocompatible photoink of claim 10, wherein the photo-absorber is selected from the group consisting of tatrazine, curcumin, anthocyanin, and combinations thereof.

12. The three-dimensional biocompatible photoink of claim 10, wherein the photo-absorber is present in a concentration of about 1 to about 5 mM.

13. The three-dimensional biocompatible photoink of claim 1, further comprising a crosslinker.

14. The three-dimensional biocompatible photoink of claim 13, wherein the crosslinker is selected from the group consisting of polyethylene glycol dithiol, dithiothreitol, and combinations thereof.

15. The three-dimensional biocompatible photoink of claim 13, wherein the crosslinker is present in an amount of about 3 to about 15 mM.

16. The three-dimensional biocompatible photoink of claim 1, further comprising live tissue cells.

17. The three-dimensional biocompatible photoink of claim 16, wherein the live tissue cells comprise normal human astrocytes.

18. The three-dimensional biocompatible photoink of claim 1, further comprising an RGD protein sequence.

19. A three-dimensional biocompatible photoink, comprising:
a biocompatible polymer having at least one covalently bonded peptide;
a photoinitiator;
a photo-absorber;
a crosslinker;
live tissue cells; and
a protein sequence.

20. The method of claim 19, wherein the protein sequence is an RGD protein sequence including a cysteine amino acid.

21. A three-dimensional biocompatible photoink, comprising:
a biocompatible polymer having at least one covalently bonded peptide, including at least one of polyethylene glycol norbornene and hyaluronic acid norbornene;
a photoinitiator selected from the group consisting of lithium phenyl-2,4,6-trimethylbenzoulphosphate, 1-hydroxy cyclohexyl phenyl ketone, 2-hydroxy-2-methyl-1-phenyl-1 propanone, 2-hydroxy-1-[4-(2-hydroxyethoxy) phenyl]-2-methyl-1-propanone, and combinations thereof;
a photo-absorber selected from the group consisting of tatrazine, curcumin, anthocyanin, and combinations thereof; and
a crosslinker selected from the group consisting of polyethylene glycol dithiol, dithiothreitol, and combinations thereof.

22. The three-dimensional biocompatible photoink of claim 21, further comprising at least one of a protein sequence and live tissue cells.

23. The three-dimensional biocompatible photoink of claim 22, wherein the protein sequence is an RGD protein sequence that includes a cysteine amino acid.

24. The three-dimensional biocompatible photoink of claim 21, further comprising an RGD protein sequence that includes a cysteine amino acid, and live tissue cells.
